(19) United States
(12) Patent Application Publication
(10) Pub. No.: US 2023/0000431 A1
(43) Pub. Date: Jan. 5, 2023

(54) SYSTEM AND METHOD FOR MONITORING A CONSCIOUSNESS-ALTERING THERAPEUTIC SESSION

(71) Applicant: Mind Medicine, Inc., New York, NY (US)

(72) Inventors: Martin MAJERNIK, Senec (SK); Daniel R. KARLIN, New York, NY (US); Robert BARROW, Madison, WI (US); Peter DUBEC, Bratislava (SK)

(73) Assignee: Mind Medicine, Inc., New York, NY (US)

(21) Appl. No.: 17/839,374

(22) Filed: Jun. 13, 2022

Related U.S. Application Data
(60) Provisional application No. 63/216,683, filed on Jun. 30, 2021.

(51) Int. Cl. A61B 5/00; A61B 5/11; A61B 5/0205; A61B 5/16

(52) U.S. Cl. CPC A61B 5/4848; A61B 5/681; A61B 5/4803; A61B 5/1113; A61B 5/0205; A61B 5/165; A61B 5/1118; A61B 5/6898; A61B 5/74; A61B 2562/0204; A61B 2562/0219; A61B 5/021

(57) ABSTRACT

A system for monitoring patients during a consciousness-altering therapeutic treatment session including a data collection module in electronic communication with network servers for storage of data on non-transitory computer readable media for monitoring a patient's well-being during and after the treatment session. A method of using the system in treating a patient, by continuously, continually, or at the healthcare professionals discretion monitoring the well-being of the patient during a consciousness-altering therapeutic treatment session through one or more wearable devices and a patient mobile device in electronic communication with a facilitator mobile device, and continuously monitoring the well-being of the patient after the treatment session with the wearable device.

### BACKGROUND OF THE INVENTION

### 1. Technical Field

The present invention relates to devices and methods for monitoring patients in the course of the administration of a prescription therapeutic that is known to have consciousness altering effects requiring monitoring for safety and efficacy.

### 2. Background Art

There are many different prescription therapeutics that have consciousness-altering effects that require monitoring for safety and efficacy. One particular group of therapeutics having these effects are the class commonly referred to as psychedelics. Psychedelics are substances that are capable of inducing subjective effects such as a dream-like alteration of consciousness, affective changes, enhanced introspective abilities, visual imagery, pseudo- and true hallucinations, synesthesia, altered temporal and spatial perception, mystical-type experiences, disembodiment and ego dissolution (Liechti, 2017; Passie, Halpern, Stichtenoth, Emrich & Hintzen, 2008). Psychedelics have been used in isolation and to assist psychotherapy for many indications including anxiety, depression, addiction, personality disorder and others and can also be used to treat other disorders such as cluster headache and migraine and others (Passie et al., 2008; Hintzen et al., 2010; Nichols, 2016; Liechti, 2017).

Currently, during a treatment session, patients are administered a consciousness-altering therapeutic such as a psychedelic and are continuously monitored by medical professionals throughout its duration of action. Patients often listen to music, are provided with eyeshades, and are encouraged to stay introspective and open to feelings, and attentive to thoughts and memories. Psychotherapy can be provided before, during, and/or after a treatment session (Schenberg, Front Pharmacol. 2018; 9: 733).

With a treatment session lasting between 30 mins and 12 hours, a medical professional is limited in how many treatments can be provided in a day. There are many people who might sustain benefit from such a psychotherapy, and the availability of treatment is limited by the time required of the medical professionals in monitoring the patients, and the limited number of appropriately qualified and trained medical professionals.

Patient monitoring systems are any devices that allow healthcare providers to monitor aspects of a patient's health. Usually, the devices include a sensor to detect a particular health parameter and wiring/electronics to relay the information to a processing unit and screen to display the parameter reading. The devices can include ECG machines, thermometers, pulse oximeters, capnography machines, and sphygmomanometers.

Remote patient monitoring systems allow healthcare providers to monitor a patient remotely, i.e., outside of a hospital or physician's office. These devices collect data about a patient, store the data, and can transmit the data to a healthcare provider. The healthcare provider can review the data collected and make suggestions to the patient in treatment. Devices include health and fitness trackers, wearable heart monitors, and glucose monitors.

Remote patient monitoring provides several advantages. Providing real-time health data to a healthcare provider allows the healthcare provider to be aware of their patient's current health status, be aware of patient's safety, and make informed decisions and adjustments in dosing or type of medication. There are fewer in-person visits to a doctor's office required for the patient. The patient can often view their health results in a smart phone application so they can be aware of their health. It also allows for healthcare providers to monitor more patients at once.

There remains a need for a method of monitoring patients receiving consciousness-altering therapeutics such as psychedelics to allow more patients to receive treatment.

### SUMMARY OF THE INVENTION

The present invention provides for a system for monitoring patients during a consciousness-altering therapeutic treatment session including a data collection module in electronic communication with network servers for storage of data on non-transitory computer readable media for monitoring a patient's well-being during and after the treatment session.

The present invention generally provides for a method of using the system in treating a patient, by continuously, continually, or at the healthcare professionals discretion monitoring the well-being of the patient during a consciousness-altering therapeutic treatment session through one or more wearable devices and a patient mobile device in electronic communication with a facilitator mobile device, and continuously monitoring the well-being of the patient after the treatment session with the wearable device.

### DESCRIPTION OF THE DRAWINGS

Other advantages of the present invention are readily appreciated as the same becomes better understood by reference to the following detailed description when considered in connection with the accompanying drawings wherein:

FIG. 1 is a flow chart of movement of information within the system;

FIG. 2 is a further detailed diagram of components and data of the system; and

FIG. 3 is a screenshot of a facilitator pre-session checklist.

### DETAILED DESCRIPTION OF THE INVENTION

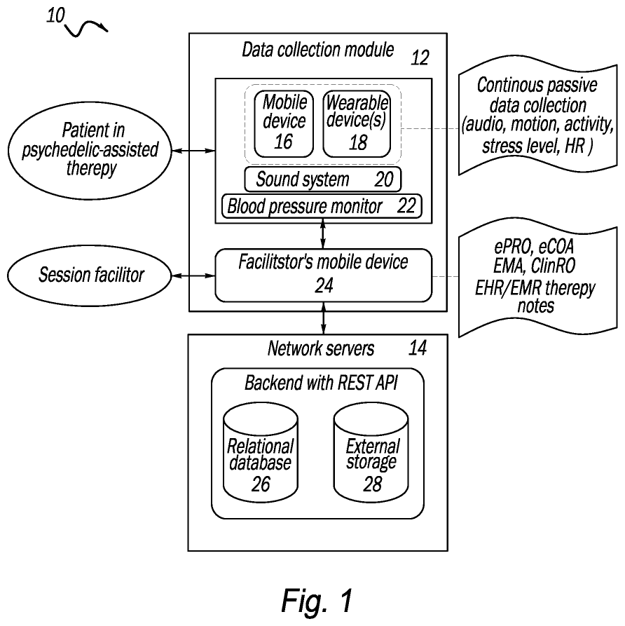

The present invention generally provides for a system 10, shown in FIG. 1, for monitoring the well-being of patients during a consciousness-altering therapeutic treatment session that includes a data collection module 12 in electronic communication with network servers 14 for storage of data on non-transitory computer readable media. The system 10 allows for continuous monitoring of patients both at a treatment center as well as after a treatment session at home or otherwise remote from the treatment center. The system 10 also enhances the ability of the administering healthcare professional, in combination with clinical judgment, to determine when the patient is no longer acutely affected by the therapeutic agent and the treatment session can be ended.

Most generally, the system 10 monitors the well-being of a patient, which includes how they are reacting to a consciousness-altering therapeutic treatment, both during a treatment session and immediately after a treatment session has ended. The system 10 can monitor adverse events (including negative emotional valence and anxiety) that can require acute attention or care to the patient, extent of a treatment session with consciousness altering effects, or change of therapeutic approach.

More specifically, the data collection module 12 includes patient-associated devices including a patient mobile device 16 (such as a smartphone or tablet), at least one wearable device 18 (such as a smartwatch or fitness band), a sound system 20, and a blood pressure monitor 22. The patient mobile device(s) 16 and wearable device(s) 18 provide continuous passive data collection (such as audio, motion, activity, stress level, heart rate, and combinations thereof).

The data collection module 12 also includes a facilitator mobile device 24 in electronic communication with the patient-associated devices that a session facilitator can use to monitor the patient. The facilitator mobile device 24 provides audio and video feed, ePRO, eCOA, EMA, ClinRO, EHR/EMR, cognitive tasks and therapy notes. The facilitator mobile device 24 can include an application (or “app”) that provides guidance through a treatment session.

The network servers 14 further include a backend with API including a relational database 26 and external storage 28.

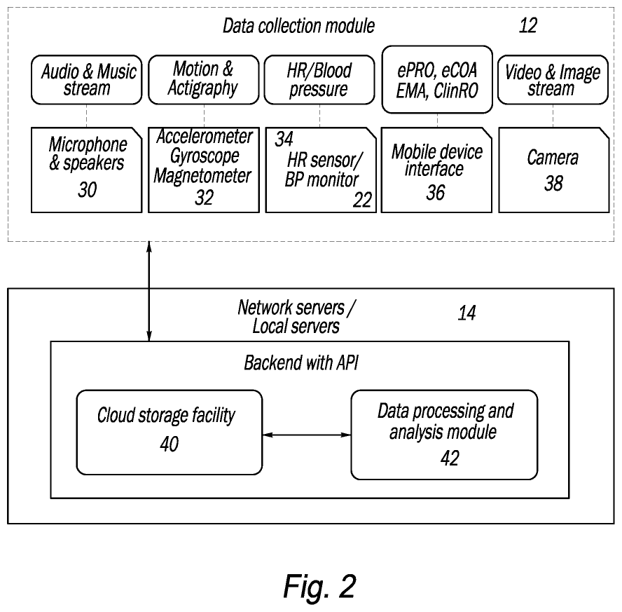

FIG. 2 shows the type of data collected in the system 10. The data collection module 12 can collect audio and music stream data with a microphone and speakers 30, motion and actigraphy with an accelerometer, gyroscope, and magnetometer 32, heart rate and blood pressure with a heart rate sensor 34 and blood pressure monitor 22, ePRO, eCOA, EMA, or Clin RO with the facilitator mobile device interface 36, and video and image stream with a camera 38. The network or local servers 14 can include a cloud storage facility 40 and data processing and analysis module 42.

The consciousness-altering therapeutics can be any therapeutic that is known to have consciousness-altering effects requiring monitoring for safety and efficacy. The consciousness-altering therapeutics used in the treatment sessions in the present invention can be, but are not limited to, the psychedelics lysergic acid diethylamide (LSD), psilocybin, psilocin, mescaline, 5-methoxy-N,N-dimethyltryptamine (5-MeO-DMT), dimethyltryptamine (DMT), 2,5-dimethoxy-4-iodoamphetamine (DOI), 2,5-dimethoxy-4-bromoamphetamie (DOB), ibogaine, ketamine, salts thereof, tartrates thereof, solvates thereof, isomers thereof, analogs thereof, homologues thereof, or deuterated forms thereof. The consciousness-altering therapeutics can be used in treating any disease or condition.

The present invention generally provides for a method of using the system 10 in treating a patient, by continuously monitoring the well-being of the patient during a treatment session through a wearable device 18 and patient mobile device 16 in electronic communication with a facilitator mobile device 24, and continuously monitoring the well-being of the patient after the treatment session with the wearable device 18.

More specifically, the method includes the steps of collecting baseline data from a patient with the facilitator mobile device 24, providing the patient with a wearable device 18, a patient mobile device 16, and a blood pressure monitor 22, dosing the patient with a consciousness-altering therapeutic, a facilitator performing a treatment session, continuously monitoring the patient with the wearable device 18 and patient mobile device 16, performing a post-session assessment on the patient, and monitoring the patient post-session with the wearable device 18. Each of these steps are further described in the user flows below, as well as additional steps that can be included in the method.

The following details how the system 10 is used. It should be understood that individual steps and components of all of the described user flow(s) can be changed, omitted and replaced by an alternative.

User Flow: Session Facilitator

A patient is diagnosed, a psychedelic-assisted therapy is proposed to him, and the patient agrees. The patient is scheduled for their first treatment visit, and the patient comes into the treatment facility.

A hardware and system check is performed. The facilitator comes in the session with a pre-calibrated and pre-set device toolkit, including the facilitator's mobile device 24, the patient is given the wearable device 18 of a smart watch, the patient mobile device 16 (smart phone), and a blood pressure monitor 22. The facilitator makes sure the devices are charged, makes sure the devices are paired/synchronized, makes sure the applications on the patient's devices are running/recording [status on screens], and makes sure the internet connection is working [status on screens].

The facilitator has an introductory chat with the patient about what is going to happen during the sitting.

The facilitator sets up the devices on the patient by placing the smart watch on the patient's dominant hand and asking the patient to place the smart phone in the pocket, pouch, or on the table nearby. These devices monitor the patient's behavior and physiological status from this moment throughout the session (as well as after the session).

The facilitator sets up the session and can be guided step-by-step by an on-device application interface. The facilitator insets his ID (facilitator ID), selects the patient's ID from a list or adds a new one, confirms the pre-generated session ID, selects the session type (i.e. type of consciousness-altering therapeutic such as ketamine, LSD, psilocybin, ibogaine, or others listed above), selects dosing, and the facilitator makes sure that the setting is appropriate for treatment (i.e. there are no flashing lights, no loud noises, and this can be guided by the application on the facilitator mobile device 24).

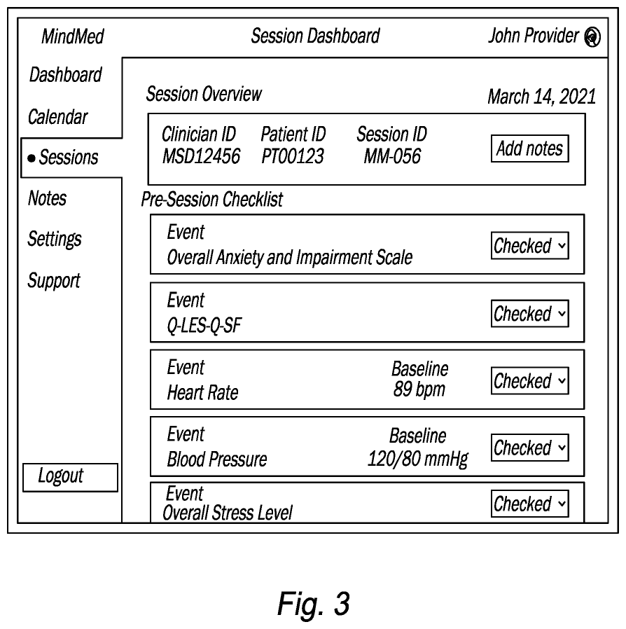

The facilitator performs a baseline assessment of the patient's psychological state and is guided step-by-step by on-device application interface, and an example of a checklist is shown in FIG. 3. The facilitator asks the patient to fill in a battery of digitized questionnaires or cognitive tasks (a specific set depends on the patient's diagnosis and treatment, e.g., for anxiety—OASIS [anxiety] Q-LES-Q-SF [quality of life]). These can be ePRO, eCOA, EMA or ClinRO. The results of these questionnaires can be visible on the facilitator mobile device 24. The facilitator asks the patient to assume a resting position, and automatically gets readings for the patient's heart rate (via smart watch), blood pressure (via Bluetooth®-enabled blood pressure monitor 22), the patient's overall stress level can be measured based on data collected so far from results of questionnaires, heart rate readings, blood pressure readings, and gathered audio (via sentiment analysis). The facilitator can ask the patient to run a urine pregnancy test and the facilitator can input the results in the application on his device 24. The facilitator can perform a drug abuse test, and the patient can look straight into a camera on the facilitator mobile device 24. The drug abuse test can be performed via computerized iris analysis, pupil analysis, facial analysis, behavioral analysis, facilitator inquiries, and/or a blood or saliva drug test. The facilitator reviews the aggregated results of the baseline check through the application on the facilitator mobile device 24.

Next, there is a baseline check with a clinician. If the facilitator is actually the clinician (on site), the facilitator has a final discussion with the patient on site and the conversation is recorded for sentiment analysis (i.e., not the content, but analysis of the patient's voice pitch, word cadence, emotional undertones). If the clinician is not on site, the patient is asked to have a video-call with the clinician via the facilitator mobile device 24.

The facilitator then administers a dose of the consciousness-altering therapeutic to the patient.

The treatment session takes place, and all steps are guided by the application on the facilitator's mobile device 24. The facilitator asks the patient to lie down or assume another comfortable position. The facilitator offers a blindfold and Bluetooth®-enabled headphones with a selection of audio tracks (e.g., ambient sounds, white noise, sounds of nature, various music styles). The facilitator encourages the patient to stay in this position throughout the session, providing support and/or therapeutic guidance. Throughout the session, the patient is continuously monitored by the wearable device 18 (smart watch) and patient mobile device 16 (smart phone). The following patient-centric data types are collected continuously: heart rate readings from dominant hand, audio masked for content processing only voice sentiment, voice pitch, and word cadence, speech sentiment, motion (gyroscope, accelerometer, orientation, elevation) from wrist of dominant hand and from waist/hip, and overall stress level (composite). The facilitator is able to observe individual metrics in real time. The facilitator receives a notification if any of the metrics differ significantly from the pre-dosing baseline readings. The facilitator is able to make a note in-app on his device 24 and can tag critical events in-session. Notes and readings from physiological and behavioral data are time-stamped and paired, along with the music the patient is listening to. The facilitator is able to review session history. The application on the facilitator mobile device 24 offers information about the length of the session [time since session ID input], estimated duration of the session (given the drug and dose), estimated end of the session (given the drug and dose), estimated intensity of administered therapeutic (given the drug and dose), and estimated time period for consciousness-altering therapeutic experience stage/phase (given the drug and dose) for early stage, onset, peak and comedown.

In a post-session assessment, the facilitator asks the patient to assume a resting position so the facilitator can assess their resting state by heart rate (via smart watch), blood pressure (via Bluetooth®-enabled blood pressure monitor 22), and the patient's overall stress level can be measured based on data collected so far from results of questionnaires, heart rate readings, blood pressure readings, and gathered audio (via sentiment analysis). All notes generated by the clinician are digitally processed, and highlights and described critical events from the therapeutic session are automatically extracted and visually presented with a timeline. The facilitator reviews aggregated results of the baseline check in-app on his device 24.

There is a post-session release check with a clinician. If the clinician is the facilitator (on site), the patient has a final discussion with the clinician on site, and the conversation is recorded for sentiment analysis (i.e., not the content, but pitch, word cadence, emotional undertones). If the clinician is not on site, the patient is asked to have a video-call with the clinician.

If the patient is considered clinically stable, the facilitator digitally signs a session report, the patient digitally signs a session report, and the patient and facilitator receive the report digitally to their email addresses. If the patient is not considered clinically stable, the facilitator asks the patient to stay in-session and the post-session release check is repeated minutes later.

At the end of a session, the facilitator collects the facilitator's mobile device 24, the blood pressure monitor 22, and the provisioned smart phone (patient mobile device 16). The facilitator releases the patient with the provisioned wearable device 18 (smart watch) for continuous monitoring of heart rate and overall activity, and a link to BYOD mobile application for reporting adverse events. Post-session, the facilitator is able to review and share automatically generated session reports, add written reports to automatically generated reports, review session highlights [tagged critical events in session—e.g., moment of elevated heart rate/stress or target notes and associated metadata], and review session history.

User Flow: Clinician

If the clinician is the session facilitator (i.e., clinician is in-session), the user flow for the session facilitator applies above. If the clinician is not the session facilitator (i.e., the clinician is not in-session), they have an additional user flow.

Pre-session, the clinician receives aggregated results of baseline assessment of a patient including results of questionnaires, the patient's heart rate readings, the patient's blood pressure readings, and the patient's overall level of stress level, drug test results, and pregnancy test results. The clinician receives a notification for a pre-session video call with the patient (supported by facilitator on site).

The clinician initiates the video call with the patient and signs off pre-session assessment and dosing.

During the session, the clinician receives notifications of critical events during the session and can be reached via video-call at any time by the facilitator and/or patient.

Post-session, the clinician receives aggregated results of post-session assessment of the patient including the patient's heart rate readings, the patient's blood pressure readings, and the patient's overall level of stress level. Aggregated notes and session highlights. The clinician receives notification for post-session release video call with the patient (supported by facilitator on site). The clinician initiates the video call with the patient and signs off the patient's release if they consider the patient clinically stable. Post-session, the clinician is able to review and share automatically generated session reports, add written a report to automatically generated reports, review session highlights [tagged critical events in session—e.g., moment of elevated heart rate/stress or target notes and associated metadata], and review session history.

User Flow: Patient

The patient is diagnosed, a therapy utilizing a consciousness-altering therapeutic is proposed to him, and the patient agrees. The patient is scheduled for their first treatment visit. The patient comes in and has a baseline assessment and an introductory discussion with the facilitator/clinician about what is going to happen during the session.

The patient is provisioned with the wearable device 18 (a smart watch) and the patient mobile device 16 (a smart phone to place in a pocket, pouch, or on a table nearby).

The patient takes baseline measurements to assess his current state. The patient fills in a battery of digitized questionnaires (a specific set depends on the patient's diagnosis and treatment). These can be ePRO, eCOA, EMA or ClinRO. The patient is asked to assume a resting position so the facilitator can assess their resting state by heart rate (via smart watch), and blood pressure (via Bluetooth®-enabled blood pressure monitor 22). The patient's overall stress level can be measured based on data collected so far from results of questionnaires, heart rate readings, blood pressure readings, and gathered audio (via sentiment analysis). The patient can run a urine pregnancy test and provide the results to a facilitator. The patient is asked to look straight into a camera 38 on the facilitator's mobile device 24. The drug abuse test can be performed via iris analysis, iris analysis logic, facial analysis, facilitator inquiries, and/or a blood or saliva drug test.

During a baseline check with a clinician, if the clinician is the facilitator (on site), the patient has a final discussion with the clinician on site, and the conversation is recorded for sentiment analysis (i.e., not the content, but pitch, word cadence, emotional undertones). If the clinician is not on site, the patient is asked to have a video-call with the clinician.

The patient then takes a dose of the consciousness-altering therapeutic provided by the facilitator.

The session takes place, and the patient is asked to lie down or assume another comfortable position. The patient can take a blindfold. The patient can take Bluetooth®-enabled headphones with a selection of audio tracks (e.g., ambient sounds, white noise, sounds of nature, various music styles). The patient is encouraged to stay in this position throughout the session, but is free to move around in any way, or reach out to the facilitator if needed. Throughout the session, the patient can be therapeutically guided, and the patient is continuously monitored by smart watch and smart phone. The following patient-centric data types are collected continuously: heart rate (dominant hand), audio masked for content (processing only voice sentiment, voice pitch, and word cadence), motion (gyroscope, accelerometer, orientation, elevation) from wrist of dominant hand and from waist/hip, and overall stress level (composite).

In a post-session assessment, the patient Is asked to assume a resting position so the facilitator can assess their resting state by heart rate (via smart watch) and blood pressure (via Bluetooth®-enabled blood pressure monitor 22). The patient's overall stress level is measured based on data collected so far from results of questionnaires, heart rate readings, blood pressure readings, and gathered audio (via sentiment analysis). In a post-session check with a clinician, if the clinician is the facilitator (on site), the patient has a final discussion with the clinician on site, and the conversation is recorded for sentiment analysis (i.e., not the content, but pitch, word cadence, emotional undertones). If the clinician is not on site, the patient is asked to have a video-call with the clinician. If the patient is considered clinically stable, the patient is asked to digitally sign a psychedelic session report and the patient receives the report digitally to his email address. If the patient is not considered clinically stable, the patient is asked to stay in-session and the post-session check is repeated minutes later.

The patient leaves the session with the provisioned wearable device 18 (smart watch) for continuous monitoring of heart rate and overall activity. The patient downloads a BYOD app for reporting of adverse events.

TABLE 1 provides feature-level specification for components in the system 10 and associated applications:

TABLE 1

| Device/App | Sampling rate |
|---|---|
| Wearable(s) or smartwatch(es) | ** |
| | configurable |
| Smartphone(s) | configurable |
| | configurable |
| Smartphone(s) + Wearable(s) or | variable sampling rate** |
| smartwatch (composite measurements)* | |
| Facilitator's device | configurable |
| Pregnancy test | n/a |
| Participant questionnaires | 1 time pre session, to judge |
| | patient's clinical stability |

*measurements are calculated based on a composite between the smartphone and the wearable.
**measurements have a variable sampling rate, depending on the recognized activity, therefore timestamps are irregular but without data loss.

Additional metadata are also collected, relative to the device state, errors, battery levels, etc. Composite motion collected from the smartphone and wearable are signals that are processed streams of data, automatically calculated by the operating system of the devices, which contain redundant information with respect to the sensor data of the devices (accelerometer, gyroscope, magnetometer).

The following is a summary of the data type categorized per reading phase.

Baseline assessment only reading: Urine pregnancy test (facilitator's input), experimental: Drug abuse iris analysis, iris analysis logic, or facial analysis), and eCOA ePRO.

Continuous monitoring (in-session): heart rate (dominant hand), audio, motion (gyroscope, accelerometry, elevation, activity), stress level (composite), and facilitators'/clinician notes.

Baseline and release assessment readings: blood pressure monitor, heart rate reading, and video call.

Continuous monitoring (post-session): heart rate (dominant hand), motion ((gyroscope, accelerometry, elevation, activity), and reported adverse events.

The following are device and data management protocols.

Data recording protocol on smartphone: The application on the phone records continuously, from the moment a patient is enrolled into the application until the battery is depleted, the device is shut down, or the patient is logged out of the device. The application records also while charging. After device restart, triggered either by the user or because of low battery, the data collection needs to be initiated manually.

Data upload protocol on smartphone: Through extensive battery profiling, it was found that uploads do not have as significant impact on battery as for instance zipping files or writing to disk. The system has configurable data saving and data upload periods. The system keeps recorded data backed on the device until the batch is sent successfully and deletes the data batch only after successful upload is confirmed. Phone-to-server upload is executed via Wi-Fi and/or via cellular data program. The system is optimized for Wi-Fi as the main upload channel. If Wi-Fi is not available (or of unstable signal quality), the system sends data batches via cellular data program.

Recording protocol on wearable: The application on the watch records continuously, from the moment a patient is enrolled into the application until the battery is depleted or the device is shut down. The application records also while charging. After device restart, triggered either by the user or because of low battery, the data collection needs to be initiated manually.

Data upload protocol on wearable: The system has configurable data saving and data upload periods. The system keeps recorded data backed on the device until the batch is sent successfully and deletes the data batch only after successful upload is confirmed. Watch-to-server upload is executed via Bluetooth® connection to the paired phone. In case the paired phone is out of reach, the watch has stored credentials to the Wi-Fi network used by the phone and will attempt to upload autonomously. The system is optimized for Wi-Fi as the main upload channel. If Wi-Fi is not available (or of unstable signal quality), the system sends data batches via cellular data program.

Device charging protocol: The system and all devices are optimized for overnight or between-session charging, as most of the therapeutic sessions utilizing consciousness-altering occur during the day. Furthermore, it was desired to align the required device management with day shifts of a facility's clinical staff. This simplifies third-party annotation of patient physiological and psychological state and well-being. However, the system can be modified to fit differing charging requirements or study designs, as it is expected that in future use cases the device charging schedule would need to align with the patient's incidence (e.g., withdrawal or clinical stability).

The system 10 is actively sending multiple alerts to the backend. None of these alerts are visible to the on-site device manager or patient. System alerts concern crash analytics, system monitoring, data upload failures, network failures, and devices being switched off.

The system 10 can also provide additional features. The system 10 can provide automatic detection of mood swings, early onset of panic attacks, and other psychological states and behavioral correlates of consciousness altering effects of the administered therapeutic. The system 10 can provide continuous monitoring and real-time detection of adverse events related to consciousness altering treatment (in-session and between sessions). There can be automatic analysis of the clinician's/facilitator's notes and the ability to automatically identify, extract, and present critical moments, comment, and highlights of the treatment session. The system 10 can automatically create patient specific and/or session-specific statistics. The system 10 can provide monitoring for signs of abuse or dependence. The system 10 can provide an automatic drug provisioning system. The system 10 can include a dosing administration tracking system. The system 10 can provide automatic detection of an inappropriate treatment setting. The system 10 can provide a digitized clinician, facilitator, and site certification system. The system 10 can provide a treatment management system. The system 10 can provide session scheduling, digital medical history, and an audit trail interface.

There are several advantages to the system 10 of the present invention. A clinician is no longer needed to run a treatment session, but rather a certified nurse or other facilitator can run a treatment session because the clinician can remotely monitor the data and supervise the treatment session. If anything serious occurs during a treatment session, the facilitator can review the data with the clinician quickly. This allows the clinician to monitor multiple patients at the same time instead of just a single patient. The data collected also allows the clinician to predict a serious event and this gives them time to adjust the treatment or intervene. After a treatment session, if any serious effects are present or if a serious event is predicted, the clinician can review the data that is collected while the patient is at home and intervene if necessary. Collecting data long term on patients can be used in building databases of patients and predicting results in new patients based on similar patients in the database.

Providing real-time health data to the clinician allows the clinician to be aware of the patient's current health status, be aware of the patient's safety, and make informed decisions and adjustments in dosing or type of medication. Fewer in-person visits to a doctor's office are required for the patient. The patient can view their health results in a smart phone application so they can be aware of their health.

The invention is further described in detail by reference to the following experimental examples. These examples are provided for the purpose of illustration only and are not intended to be limiting unless otherwise specified. Thus, the invention should in no way be construed as being limited to the following examples, but rather, should be construed to encompass any and all variations which become evident as a result of the teaching provided herein.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A system for monitoring patients during a consciousness-altering therapeutic treatment session comprising:
a data collection module in electronic communication with network servers for storage of data on non-transitory computer readable media for monitoring a patient's well-being during and after the treatment session.

2. The system of claim 1, wherein said data collection module includes a patient mobile device, at least one wearable device, a sound system, and a blood pressure monitor.

3. The system of claim 2, wherein said patient mobile device is chosen from the group consisting of a smartphone and tablet, and said at least one wearable device is chosen from the group consisting of a smart watch and a fitness band.

4. The system of claim 2, wherein said data collection module includes a facilitator mobile device in electronic communication with said patient mobile device, said at least one wearable device, said sound system, and said blood pressure monitor.

5. The system of claim 4, wherein said facilitator mobile device provides audio and video feed, ePRO, eCOA, EMA, ClinRO, EHR/EMR, cognitive tasks, and therapy notes.

6. The system of claim 4, wherein said facilitator mobile device includes an application stored on non-transitory computer readable media for guiding the facilitator through the treatment session.

7. The system of claim 1, wherein said network servers include a backend with API including a relational database and external storage.

8. The system of claim 1, wherein said data collection module collects audio and music stream data with a microphone and speakers, motion and actigraphy with an accelerometer, gyroscope, and magnetometer, heart rate and blood pressure with a heart rate sensor and said blood pressure monitor, ePRO, eCOA, EMA, or ClinRO with said facilitator mobile device, and video and image stream with a camera.

9. The system of claim 1, wherein said consciousness-altering therapeutic is chosen from the group consisting of lysergic acid diethylamide (LSD), psilocybin, psilocin, mescaline, 5-methoxy-N,N-dimethyltryptamine (5-MeO-DMT), dimethyltryptamine (DMT), 2,5-dimethoxy-4-iodoamphetamine (DOI), 2,5-dimethoxy-4-bromoamphetamie (DOB), ibogaine, ketamine, salts thereof, tartrates thereof, solvates thereof, isomers thereof, analogs thereof, homologues thereof, and deuterated forms thereof.

10. The system of claim 1, wherein said system provides a feature chosen from the group consisting of automatic detection of mood swings, early onset of panic attacks, and other psychological states and behavioral correlates of consciousness altering effects of the administered therapeutic, continuous monitoring and real-time detection of adverse events related to consciousness altering treatment, automatic analysis of the clinician's/facilitator's notes and the ability to automatically identify, extract, and present critical moments, comment, and highlights of the treatment session, automatically create patient specific and/or session-specific statistics, monitoring for signs of abuse or dependence, an automatic drug provisioning system, a dosing administration tracking system, automatic detection of an inappropriate treatment setting, a digitized clinician, facilitator, and site certification system, a treatment management system, session scheduling, digital medical history, and an audit trail interface, and combinations thereof.

11. A method of using a monitoring system in treating a patient, including the steps of:
continuously, continually, or at the healthcare professionals discretion monitoring the well-being of the patient during a consciousness-altering therapeutic treatment session through one or more wearable devices and a patient mobile device in electronic communication with a facilitator mobile device; and
continuously monitoring the well-being of the patient after the treatment session with the wearable device.

12. The method of claim 11, wherein said continuously, continually, or at the healthcare professionals discretion monitoring the well-being of the patient during a consciousness-altering therapeutic treatment session step is further defined as collecting baseline data from the patient with a facilitator mobile device, providing the patient with a wearable device, a patient mobile device, and a blood pressure monitor, dosing the patient with a consciousness-altering therapeutic, a facilitator performing a treatment session, continuously monitoring the patient with the wearable device and patient mobile device, and performing a post-session assessment on the patient.

13. The method of claim 12, further including, after said collecting baseline data step, the step of a clinician checking the baseline data and recording a conversation with the patient.

14. The method of claim 12, wherein said facilitator performing a treatment session step is further defined as an application stored on non-transitory computer readable media on the facilitator mobile device guiding the facilitator through the treatment session.

15. The method of claim 12, further including the step of notifying the facilitator if any metrics differ significantly from the baseline data.

16. The method of claim 12, further including the step of the facilitator making notes and tagging critical events in session in the application.

17. The method of claim 12, further including the step of the application providing information about the length of the session, estimated duration of the session, estimated end of the session, estimated intensity of administered therapeutic, and estimated time period for consciousness-altering therapeutic experience stage/phase for early stage, onset, peak and comedown.

18. The method of claim 12, wherein said performing a post-session assessment step is further defined as the facilitator assessing the patient's resting state by assessing heart rate, blood pressure, and overall stress levels, digitally processing all notes generated during the session, and automatically extracting and visually presenting highlights and critical events with a timeline on the facilitator mobile device.

19. The method of claim 18, further including the step of performing a post-session release check with a clinician.

20. The method of claim 11, wherein said continuously monitoring the well-being of the patient after the treatment session step is further defined as continuously monitoring the patient's heart rate and overall activity with the wearable device.

21. The method of claim 11, further including the step of continuously monitoring adverse events.

22. The method of claim 11, wherein the one or more wearable devices and patient mobile devices collect passive data continuously chosen from the group consisting of audio, motion, activity, stress level, heart rate, and combinations thereof.

23. The method of claim 11, wherein the consciousness-altering therapeutic is chosen from the group consisting of lysergic acid diethylamide (LSD), psilocybin, psilocin, mescaline, 5-methoxy-N,N-dimethyltryptamine (5-MeO-DMT), dimethyltryptamine (DMT), 2,5-dimethoxy-4-iodoamphetamine (DOI), 2,5-dimethoxy-4-bromoamphetamie (DOB), ibogaine, ketamine, salts thereof, tartrates thereof, solvates thereof, isomers thereof, analogs thereof, homologues thereof, and deuterated forms thereof.

24. The method of claim 11, wherein a clinician is monitoring multiple patients at the same time.

25. The method of claim 11, further including a step chosen from the group consisting of automatically detecting mood swings, early onset of panic attacks, and other psychological states and behavioral correlates of consciousness altering effects of the administered therapeutic, continuously monitoring and real-time detecting of adverse events related to consciousness altering treatment, automatically analysing the clinician's/facilitator's notes and automatically identifying, extracting, and presenting critical moments, comments, and highlights of the treatment session, automatically creating patient specific and/or session-specific statistics, monitoring for signs of abuse or dependence, providing an automatic drug provisioning system, providing a dosing administration tracking system, automatically detecting an inappropriate treatment setting, providing a digitized clinician, facilitator, and site certification system, providing a treatment management system, session scheduling, digital medical history, and an audit trail interface, and combinations thereof.

26. The method of claim 11, wherein the patient mobile device is chosen from the group consisting of a smartphone and tablet, and the at least one wearable device is chosen from the group consisting of a smart watch and a fitness band.
